(19) United States
(12) Patent Application Publication
(10) Pub. No.: US 2008/0167366 A1
(43) Pub. Date: Jul. 10, 2008

(54) Use of 2-oxo-1-pyrrolidine derivatives for the preparation of a drug

(76) Inventors: Renee Grimee, Bruxelles (BE); Henrik Klitgaard, Bruxelles (BE)

Correspondence Address: WENDEROTH, LIND & PONACK, L.L.P., 2033 K STREET N. W., SUITE 800, WASHINGTON, DC, 20006-1021, US

(21) Appl. No.: 11/819,892

(22) Filed: Jun. 29, 2007

Related U.S. Application Data
(62) Division of application No. 10/489,660, filed on Mar. 15, 2004, now abandoned.

(30) Foreign Application Priority Data
Oct. 8, 2001 (EP) 01123976.1

(51) Int. Cl. A61K 31/404; A61K 31/4015; A61P 25/16

(52) U.S. Cl. 514/418; 514/424

(57) ABSTRACT

The present invention relates to pharmaceutical compositions containing 2-oxo-1-pyrrolidine derivatives and methods for treating Parkinson's Disease in a chronic dopamine replacement therapy using L-DOPA by administering the derivatives.

The present invention relates to the use of 2-oxo-1-pyrrolidine derivatives (and in particular (S)-(−)-α-ethyl-2-oxo-1-pyrrolidineacetamide for the preparation of drugs for the curative and/or prophylactic treatment of movement disorders or dyskinesia.

Movement and other disorders due to dysfunction of the basal ganglia and related brain structures are of major socio-economic importance. Such disorders can occur as a consequence of inherited or acquired disease, idiopathic neurodegeneration or they may be iatrogenic. The spectrum of disorders is very diverse, ranging from those associated with poverty of movement (akinesia, hypokinesia, bradykinesia) and hypertonia (e.g. Parkinson's disease) to the involuntary movement disorders (hyperkinesias or dyskinesias e.g. Huntington's disease, L-DOPA-induced dyskinesia, tardive dyskinesia, progressive supernuclear palsy, multiple system atrophy, corticobasal degeneration, Wilson's disease, progressive pallidal atrophy).

Parkinson's disease and related conditions represent one of the most prevalent diseases associated with poverty of movement. Parkinsonian symptoms manifest as a syndrome of symptoms characterised by slowness of movement (bradykinesia), rigidity and/or tremor. Parkinsonian symptoms are seen in a variety of conditions, most commonly in idiopathic parkinsonism (i.e. Parkinson's Disease) but also following treatment of schizophrenia ( i.e. neuroleptic induced parkinsonism), exposure to toxins/drugs and head injury.

It is widely appreciated that the primary pathology underlying Parkinson's disease is degeneration, in the brain, of the dopaminergic projection from the substantia nigra to the striatum. This has led to the widespread use of dopamine-replacing agents (e.g. L-3,4-dihydroxyphenylalanine (L-DOPA) and dopamine agonists) as symptomatic treatments for Parkinson's disease . Such treatments have been successful in increasing the quality of life of patients suffering from Parkinson's disease. However, dopamine-replacement treatments do have limitations, especially following long-term treatment. Problems can include fluctuations (e.g. “on-off” phenomenon, wearing-off of the anti-parkinsonian efficacy of the treatment) and the appearance of a range of side-effects which manifest as abnormal involuntary movements, such as dyskinesias.

Dyskinesias, as a whole, are characterised by the development in a subject of abnormal involuntary movements. One way in which dyskinesias may arise is as a side effect of dopamine replacement therapy for parkinsonism or other basal ganglia-related movement disorders.

Many attempts have been made to identify agents that will prevent the development of, and/or treat dyskinesias although such attempts have met with limited success. There is therefore, a need to discover ways by which movement disorders and dyskinesias may be treated.

The use of levorotatory (S)-α-ethyl-2-oxo-1-pyrrolidineacetamide, also known as levetiracetam [International Nonproprietary Name] as a protective agent for the treatment and prevention of hypoxic and ischaemic type aggressions of the central nervous system is described in the European patent EP-A-0162 036. That compound can also be employed in the treatment of epilepsy, a therapeutic indication for which it has been demonstrated that its dextrorotatory enantiomer, (R)-(+)-α-ethyl-2-oxo-1-pyrrolidine-acetamide, is completely devoid of activity (A. J. GOWER et al., Eur. J. Pharmacol., 222, (1992), 193-203). That compound has also been described in European patent EP-A-0 645 139 for the treatment of anxiety.

EP-A-162 036 cited above also describes methods for preparing (S)-(−)-α-ethyl-2-oxo-1-pyrrolidine-acetamide which require the synthesis of a starting reactant obtained by resolution of the corresponding racemate. British patent GB-A-2 225 322 describes a method for preparing that compound which offers the advantage of using a natural amino acid which already has the desired stereochemical configuration as the starting material, thus dispensing with any laborious separation of the enantiomers.

2-oxo-1-pyrrolidine derivatives are described in the international patent application WO 01/62726 as well as their use as pharmaceuticals. The derivatives are particularly suited for treating neurological disorders such as epilepsy.

In continuing its research on these compounds, the Applicant has now discovered that (S)-(−)-α-ethyl-2-oxo-1-pyrrolidineacetamide and also 2-oxo-1-pyrrolidine derivatives possess therapeutic properties which render it particularly useful in the treatment and prophylaxis of movement disorders and dyskinesia.

The present invention thus concerns the use of an active compound which is a 2-oxo-1-pyrrolidine derivatives having the formula II or a pharmaceutically acceptable salt thereof.

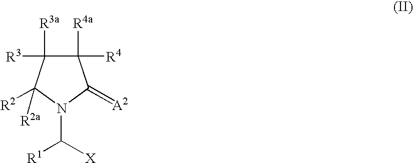

wherein

X is -CA¹NR⁵R⁶ or -CA¹OR⁷ or -CA¹-R⁸ or CN;

A¹ and A² are independently oxygen, sulfur or —NR⁹;

R¹ is hydrogen, alkyl, aryl or —CH₂—R^{1a} wherein R^{1a} is aryl, heterocycle, halogen, hydroxy, amino, nitro or cyano;

R², R³ and R⁴ are the same or different and each is independently hydrogen, halogen, hydroxy, thiol, amino, nitro, nitrooxy, cyano, azido, carboxy, amido, sulfonic acid, sulfonamide, alkyl, alkenyl, alkynyl, ester, ether, aryl, heterocycle, or an oxy derivative, thio derivative, amino derivative, acyl derivative, sulfonyl derivative or sulfinyl derivative;

R^{2a}, R^{3a} and R^{4a} are the same or different and each is independently hydrogen, halogen, alkyl, alkenyl, alkynyl or aryl;

R⁵, R⁶, R⁷and R⁹ are the same or different and each is independently hydrogen, hydroxy, alkyl, aryl, heterocycle or an oxy derivative; and

R⁸ is hydrogen, hydroxy, thiol, halogen, alkyl, aryl, heterocycle or a thio derivative; with the provisos that at least one of as R², R³, R⁴, R^{2a}, R^{3a} and R^{4a} is other than hydrogen; and that when the compound is a mixture of all possible isomers, X is —CONR⁵R⁶, A² is oxygen and R¹ is hydrogen, methyl, ethyl or propyl then substitution on the pyrollidine ring is other than mono-, di-, or tri-methyl or mono-ethyl; and that when R¹, R², R⁴, R^{2a}, R^{3a} and R^{4a} are each hydrogen, A² is oxygen and X is CONR⁵R⁶ then R³ is different from carboxy, ester, amido, substituted oxo-pyrrolidine, hydroxy, oxy derivative, amino, amino derivatives, methyl, naphthyl, phenyl optionally substituted by oxy derivatives or in the para position by an halogen atom;

for the preparation of drugs for the treatment or prophylaxis of dyskinesia.

The present invention concerns also the use of an active compound which is (S)-(−)-α-ethyl-2-oxo-1-pyrrolidineacetamide having the formula I

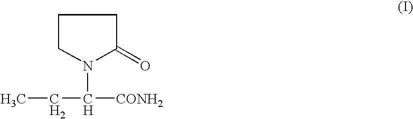

for the preparation of drugs for the treatment or prophylaxis of movement disorders or dyskinesia.

In a first aspect, the invention concerns the use of the active compound for the manufacture of a medicament for treatment and/or prophylactic treatment of dyskinesia.

The present invention also concerns a method for treating or preventing dyskinesia, comprising administering a therapeutic amount of the active compound, as described above, to a patient. In particular, it concerns a method for treating or preventing movement disorders or dyskinesia, comprising administering a therapeutic amount of (S)-(−)-α-ethyl-2-oxo-1-pyrrolidineacetamide to a patient in need.

The term “treatment” as used by the Applicant means curative treatment and prophylactic treatment.

By “curative” we mean the efficaciousness of the active compound in treating the current episode.

By “prophylactic” or “maintenance” we mean the prevention of any induction of the recurrence of episodes and the possibility to de-prime the manifestation of dyskinesia.

By “movement disorder”, we mean in particular movement disorder associated with a poverty of movement and more particularly to the treatment of parkinsonism, a medical condition characterised by akinesia, hypokinesia or bradykinesia and also conditions characterised by hypertonia. Such disorders include Wilson's disease, progressive supranuclear palsy, and in particular Parkinson's disease and other forms of parkinsonism.

By “dyskinesia” we mean the development in a subject of abnormal involuntary movements. This appears in patients with Huntington's disease, in Parkinson's disease patients exposed to chronic dopamine replacement therapy, and in Schizophrenia patients exposed to chronic treatment with neuroleptics.

The inventors have established that the use of active compounds alone significantly reduces the problems associated with conventional therapies. For instance, side-effects such as abnormal involuntary movements (dyskinesias) induced by conventional therapies do not develop, or develop to a lesser extent, when active compounds are used in combination with these therapies to treat parkinsonism, schizophrenia and Huntington's diseases, and in particular parkinsonism.

The invention is based upon our studies relating to the use of active compounds to alleviate significantly L-DOPA-induced dyskinesias in a non-human primate model of Parkinson's disease.

The 2-oxo-1-pyrrolidine derivatives having the formula II are described in the international patent application WO 01/62726, the content of the application is incorporated by reference.

For the active compounds, in the definitions set forth below, unless otherwise stated, R¹¹ and R¹² are the same or different and each is independently amido, alky, alkenyl, alkynyl, acyl, ester, ether, aryl, aralkyl, heterocycle or an oxy derivative, thio derivative, acyl derivative, amino derivative, sulfonyl derivative, or sulfinyl derivative, each optionally substituted with any suitable group, including, but not limited to, one or more moieties selected from lower alkyl or other groups as described below as substituents for alkyl.

The term “oxy derivative”, as used herein is defined as including —O—R¹¹ groups wherein R¹¹ is as defined above except for “oxy derivative”. Non-limiting examples are alkoxy, alkenyloxy, alkynyloxy, acyloxy, oxyester, oxyamido, alkylsulfonyloxy, alkylsulfinyloxy, arylsulfonyloxy, arylsulfinyloxy, aryloxy, aralkoxy or heterocyclooxy such as pentyloxy, allyloxy, methoxy, ethoxy, phenoxy, benzyloxy, 2-naphthyloxy, 2-pyridyloxy, methylenedioxy, carbonate.

The term “thio derivative” as used herein, is defined as including —S—R¹¹ groups wherein R¹¹ is as defined above except for “thio derivative”. Non-limiting examples are alkylthio, alkenylthio, alkynylthio and arylthio.

The term “amino derivative” as used herein, is defined as including —NHR¹¹ or —NR¹¹R¹² groups wherein R¹¹ and R¹² are as defined above. Non-limiting examples are mono- or di-alkyl-, alkenyl-, alknyl- and arylamino or mixed amino.

The term “acyl derivative” as used herein, represents a radical derived from carboxylic acid and thus is defined as including groups of the formula R¹¹—CO—, wherein R¹¹ is as defined above and may also be hydrogen. Non-limiting examples are formyl, acetyl, propionyl, isobutyryl, valeryl, lauroyl, heptanedioyl, cyclohexanecarbonyl, crotonoyl, fumaroyl, acryloyl, benzoyl, naphthoyl, furoyl, nicotinoyl, 4-carboxybutanoyl, oxalyl, ethoxalyl, cysteinyl, oxamoyl.

The term “sulfonyl derivative” as used herein, is defined as including a group of the formula —SO₂—R¹¹, wherein R¹¹ is as defined above except for “sulfonyl derivative”. Non-limiting examples are alkylsulfonyl, alkenylsulfonyl, alkynylsulfonyl and arylsulfonyl.

The term “sulfinyl derivative” as used herein, is defined as including a group of the formula —SO—R¹¹, wherein R¹¹ is as defined above except for “sulfinyl derivative”. Non-limiting examples are alkylsulfinyl, alkenylsulfinyl, alkynylsulfinyl and arylsulfinyl.

The term “alkyl”, as used herein, is defined as including saturated, monovalent hydrocarbon radicals having straight, branched or cyclic moieties or combinations thereof and containing 1-20 carbon atoms, preferably 1-6 carbon atoms for non-cyclic alkyl and 3-6 carbon atoms for cycloalkyl (in these two preferred cases, unless otherwise specified, “lower alkyl”). Alkyl moieties may optionally be substituted by 1 to 5 substituents independently selected from the group consisting of halogen, hydroxy, thiol, amino, nitro, cyano, thiocyanato, acyl, acyloxy, sulfonyl derivative, sulfinyl derivative, alkylamino, carboxy, ester, ether, amido, azido, cycloalkyl, sulfonic acid, sulfonamide, thio derivative, oxyester, oxyamido, heterocycle, vinyl, C1-5-alkoxy, C6-10-aryloxy and C6-10-aryl.

Preferred alkyl groups are methyl, ethyl, propyl, isopropyl, butyl, iso or ter-butyl, and 2,2,2-trimethylethyl each optionally substituted by at least one substituent selected from the group consisting of halogen, hydroxy, thiol, amino, nitro and cyano, such as trifluoromethyl, trichloromethyl, 2,2,2-trichloroethyl, 1,1-dimethyl-2,2-dibromoethyl, 1,1-dimethyl-2,2,2-trichloroethyl.

The term “alkenyl” as used herein, is defined as including both branched and unbranched, unsaturated hydrocarbon radicals having at least one double bond such as ethenyl (=vinyl), 1-methyl-1-ethenyl, 2,2-dimethyl-1-ethenyl, 1-propenyl, 2-propenyl (=allyl), 1-butenyl, 2-butenyl, 3-butenyl, 4-pentenyl, 1-methyl-4-pentenyl, 3-methyl-1-pentenyl, 1-hexenyl, 2-hexenyl, and the like and being optionally substituted by at least one substituent selected from the group consisting of halogen, hydroxy, thiol, amino, nitro, cyano, aryl and heterocycle such as mono- and di-halo vinyl where halo is fluoro, chloro or bromo.

The term “alkynyl” as used herein, is defined as including a monovalent branched or unbranched hydrocarbon radical containing at least one carbon-carbon triple bond, for example ethynyl, 2-propynyl (=propargyl), and the like and being optionally substituted by at least one substituent selected from the group consisting of halogen, hydroxy, thiol, amino, nitro, cyano, aryl and heterocycle, such as haloethynyl.

When present as bridging groups, alkyl, alkenyl and alkynyl represent straight- or branched chains, C1-12, preferably C1-4-alkylene or C2-12-, preferably C2-4-alkenylene or -alkynylene moieties respectively.

Groups where branched derivatives are conventionally qualified by prefixes such as “n”, “sec”, “iso” and the like (e.g. “n-propyl”, “sec-butyl”) are in the n-form unless otherwise stated.

The term “aryl” as used herein, is defined as including an organic radical derived from an aromatic hydrocarbon consisting of 1-3 rings and containing 6-30 carbon atoms by removal of one hydrogen, such as phenyl and naphthyl each optionally substituted by 1 to 5 substituents independently selected from halogen, hydroxy, thiol, amino, nitro, cyano, acyl, acyloxy, sulfonyl, sulfinyl, alkylamino, carboxy, ester, ether, amido, azido, sulfonic acid, sulfonamide, alkylsulfonyl, alkylsulfinyl, alkylthio, oxyester, oxyamido, aryl, C1-6-alkoxy, C6-10-aryloxy, C1-6-alkyl, C1-6-haloalkyl. Aryl radicals are preferably monocyclic containing 6-10 carbon atoms. Preferred aryl groups are phenyl and naphthyl each optionally substituted by 1 to 5 substituents independently selected from halogen, nitro, amino, azido, C1-6-alkoxy, C1-6-alkylthio, C1-6-alkyl, C1-6-haloalkyl and phenyl.

The term “halogen”, as used herein, includes an atom of Cl, Br, F, I.

The term “hydroxy”, as used herein, represents a group of the formula —OH.

The term “thiol”, as used herein, represents a group of the formula —SH.

The term “cyano”, as used herein, represents a group of the formula —CN.

The term “nitro”, as used herein, represents a group of the formula —NO₂

The term “nitrooxy”, as used herein, represents a group of the formula —ONO₂.

The term “amino”, as used herein, represents a group of the formula —NH₂.

The term “azido”, as used herein, represents a group of the formula —N₃

The term “carboxy”, as used herein, represents a group of the formula —COOH.

The term “sulfonic acid”, as used herein, represents a group of the formula —SO₃H.

The term “sulfonamide”, as used herein, represents a group of the formula —SO₂NH₂.

The term “ester” as used herein is defined as including a group of formula —COO—R¹¹ wherein R¹¹ is as defined above except oxy derivative, thio derivative or amino derivative.

The term “ether” is defined as including a group selected from C1-50- straight or branched alky, or C2-50- straight or branched alkenyl or alknyl groups or a combination of the same, interrupted by one or more oxygen atoms.

The term “amido” is defined as including a group of formula —CONH₂ or —CONHR¹¹ or —CONR¹¹R¹² wherein R¹¹ and R¹² are as defined above.

The term “heterocycle”, as used herein is defined as including an aromatic or non aromatic cyclic alkyl, alkenyl, or alkynyl moiety as defined above, having at least one O, S and/or N atom interrupting the carbocyclic ring structure and optionally, one of the carbon of the carbocyclic ring structure may be replaced by a carbonyl. Non-limiting examples of aromatic heterocycles are pyridyl, furyl, pyrrolyl, thienyl, isothiazolyl, imidazolyl, benzimidazolyl, tetrazolyl, quinazolinyl, quinolizinyl, naphthyridinyl, pyridazinyl, pyrimidinyl, pyrazinyl, quinolyl, isoquinolyl, isobenzofuranyl, benzothienyl, pyrazolyl, indolyl, indolizinyl, purinyl, isoindolyl, carbazolyl, thiazolyl, 1,2,4-thiadiazolyl, thieno(2,3-b)furanyl, furopyranyl, benzofuranyl, benzoxepinyl, isooxazolyl, oxazolyl, thianthrenyl, benzothiazolyl, or benzoxazolyl, cinnolinyl, phthalazinyl, quinoxalinyl, phenanthridinyl, acridinyl, perimidinyl, phenanthrolinyl, phenothiazinyl, furazanyl, isochromanyl, indolinyl, xanthenyl, hypoxanthinyl, pteridinyl, 5-azacytidinyl, 5-azauracilyl, triazolopyridinyl, imidazolopyridinyl, pyrrolopyrimidinyl, and pyrazolopyrimidinyl optionally substituted by alkyl or as described above for the alkyl groups. Non-limiting examples of non aromatic heterocycles are tetrahydrofuranyl, tetrahydropyranyl, piperidinyl, piperidyl, piperazinyl, imidazolidinyl, morpholino, morpholinyl, 1-oxaspiro(4.5)dec-2-yl, pyrrolidinyl, 2-oxo-pyrrolidinyl, sugar moieties (i.e. glucose, pentose, hexose, ribose, fructose, which may also be substituted) or the same which can optionally be substituted with any suitable group, including but not limited to one or more moieties selected from lower alkyl, or other groups as described above for the alkyl groups. The term “heterocycle” also includes bicyclic, tricyclic and tetracyclic, spiro groups in which any of the above heterocyclic rings is fused to one or two rings independently selected from an aryl ring, a cyclohexane ring, a cyclohexene ring, a cyclopentane ring, a cyclopentene ring or another monocyclic heterocyclic ring or where a monocyclic heterocyclic group is bridged by an alkylene group, such as quinuclidinyl, 7-azabicyclo(2.2.1)heptanyl, 7-oxabicyclo(2.2.1)heptanyl, 8-azabicyclo(3.2.1)octanyl.

In the above definitions it is to be understood that when a substituent such as R², R³, R⁴, R^{2a}, R^{3a}, R^{4a}, R⁵, R⁶, R⁷, R⁸ is attached to the rest of the molecule via a heteroatom or a carbonyl, a straight- or branched chain, C1-12-, preferably C1-4-alkylene or C2-12, preferably C2-4-alkenylene or -alkynylene bridge may optionally be interposed between the heteroatom or the carbonyl and the point of attachment to the rest of the molecule.

Preferred examples of X are —COO R⁷ or —CONR⁵R⁶, wherein R⁵, R⁶ and R⁷ are preferably hydrogen, C1-4-alkyl, phenyl or alkylphenyl.

Preferably X is carboxy or —CONR⁵R⁶, wherein R⁵ and R⁶ are preferably hydrogen, C1-4-alkyl, phenyl or alkylphenyl, especially —CONH₂.

Preferably A¹ and A² are each oxygen.

Preferably R¹ is hydrogen, alkyl, especially C1-12 alkyl, particularly lower alkyl or aryl especially phenyl.

Examples of preferred R¹ groups are methyl, ethyl, propyl, isopropyl, butyl, iso- or ter-butyl, 2,2,2-trimethylethyl each optionally attached via a methylene bridge or the same substituted by at least one halogen atom such as trifluoromethyl, trichloromethyl, 2,2,2-trichloroethyl, 1,1-dimethyl-2,2-dibromoethyl, 1,1-dimethyl-2,2,2-trichloroethyl. R¹ as ethyl is especially preferred.

Preferably R² and R²a are independently hydrogen, halogen or alkyl, especially lower alkyl.

Examples of preferred R² and R^{2a} groups are independently hydrogen, halogen or methyl, ethyl, propyl, isopropyl, butyl, iso or ter-butyl, 2,2,2-trimethylethyl or the same substituted by at least one halogen atom such as trilfluoromethyl, trichloromethyl, 2,2,2-trichloroethyl, 1.1-dimethyl-2,2-dibromoethyl, 1,1-dimethyl-2,2,2-trichloroethyl. Especially at least one and most preferably both of R² and R^{2a} are hydrogen. Preferably R^{3a}, R⁴ and R^{4a} are independently hydrogen, alkyl, especially methyl or ethyl or aryl especially phenyl or aralkyl, especially benzyl.

Examples of preferred R^{3a}, R⁴ and R^{4a} groups are independently hydrogen, halogen or methyl, ethyl, propyl, isopropyl, butyl, iso or ter-butyl, 2,2,2-trimethylethyl or the same substituted by at least one halogen atom such as trifluoromethyl, trichloromethyl, 2,2,2-trichloroethyl, 1,1-dimethyl-2,2-dibromoethyl, 1,1-dimethyl-2,2,2-trichloroethyl. Especially at least one and most preferably both of R⁴ and R^{4a} are hydrogen. R^{3a} is particularly hydrogen or alkyl, especially lower allyl and is most preferably hydrogen. Preferably R³ is hydrogen, C1-12-alkyl, especially C1-6-alkyl, each optionally substituted by one or more substituents selected from hydroxy, halogen, cyano, thiocyanato or alkoxy and attached to the ring either directly or via a thio, sulfinyl, sulfonyl, carbonyl or oxycarbonyl group and optionally, a C1-4-alkylene bridge, particularly methylene; C2-6-alkenyl or -alkynyl, especially C2-3-alkenyl or -alkynyl each optionally substituted by one or more halogens; azido; cyano: amido; carboxy; triazolyl, tetrazolyl, pyrrolidinyl, pyridyl, 1-oxidopyridyl, thiomorpholinyl, benzodioxolyl, furyl, oxazolyl, pyrimidinyl, pyrrolyl, thiadiazolyl, thiazolyl, thienyl or piperazinyl each optionally substituted by one or more substituents selected from halogen, C1-6-alkyl and phenyl and attached to the ring either directly or via a carbonyl group or a C1-4-alkylene bridge, particularly methylene; naphthyl; or phenyl, phenylalkyl or phenylalkenyl each optionally substituted by one or more substituents selected from halogen, C1-6-alkyl, C1-6 haloalkyl, C1-6-alkoxy, C1-6-alkylthio, amino, azido, phenyl and nitro and each attached to the ring either directly or via an oxy, sulfonyl, sulfonyloxy, carbonyl or carbonyloxy group and optionally additionally a C1-4-alkylene bridge, particularly methylene.

Also, preferably, R³ is C1-6-alkyl optionally substituted by one or more substituents selected from halogen, thiocyanato, azido, alkoxy, alkylthio, phenylsulfonyl; nitrooxy; C2-3-alkenyl or -alkynyl each optionally substituted by one or more halogens or by acetyl; tetrazolyl, pyridyl, furyl, pyrrolyl, thiazolyl or thienyl; or phenyl or phenylalkyl each optionally substituted by one or more substituents selected from halogen, C1-6-alkyl, C1-6 haloalkyl, C1-6-alkoxy, amino, azido, phenyl and nitro and each attached to the ring either directly or via a sulfonyloxy and optionally additionally a C1-4-alkylene bridge, particularly methylene.

Other examples of preferred R³ groups are hydrogen, halogen or methyl, ethyl, propyl, isopropyl, butyl, iso or ter-butyl, 2,2,2-trimethylethyl or the same substituted by at least one halogen atom such as trifluoromethyl, trichloromethyl, 2,2,2-trichloroethyl, 1,1-dimethyl-2,2-dibromoethyl, 1,1-dimethyl-2,2,2-trichloroethyl. R³ is especially C1-4-alkyl optionally substituted by one or more substituents selected from halogen, thiocyanato or azido; C2-5-alkenyl or -alkynyl, each optionally substituted by one or more halogens; thienyl: or phenyl optionally substituted by one or more substituents selected from halogen, C1-6-alkyl, C1-6 haloalkyl or azido.

Further examples of preferred R³ groups are C1-6 alkyl and C2-6 haloalkenyl.

Preferably R⁵ and R⁶ are independently hydrogen, methyl, ethyl, propyl, isopropyl, butyl, iso or ter-butyl, 2,2,2-trimethylethyl, especially hydrogen or methyl.

Especially at least one and most preferably both of R⁵ and R⁶ are hydrogen.

Preferably R⁷ is hydrogen, methyl, ethyl, propyl, isopropyl, butyl, iso or tert-butyl, 2,2,2-trimethylethyl, methoxy, ethoxy, phenyl, benzyl or the same substituted by at least one halogen atom such as trifluoromethyl, chlorophenyl.

Preferably R⁷ is hydrogen, methyl or ethyl especially hydrogen.

Preferably R⁸ is hydrogen, methyl, ethyl, propyl, isopropyl, butyl, iso or ter-butyl, 2,2,2-trimethylethyl, phenyl, benzyl or the same substituted by at least one halogen atom such as trifluoromethyl, chlorobenzyl.

Preferably R⁸ is hydrogen or methyl.

Combinations of one or more of these preferred compound groups are especially preferred.

A particular group of compounds of formula II (Compounds 1A) comprises those

wherein,

A2 is oxygen;

X is —CONR⁵R⁶ or —COOR⁷ or —CO—R⁸ or CN;

R¹ is hydrogen or alkyl, aryl, halogen, hydroxy, amino, nitro, cyano;

R², R³, R⁴, are the same or different and each is independently hydrogen or halogen, hydroxy, amino, nitro, cyano, acyl, acyloxy, a sulfonyl derivative, a sulfinyl derivative, an amino derivative, carboxy, ester, ether, amido, sulfonic acid, sulfonamide, alkoxycarbonyl, a thio derivative, alkyl, alkoxy, oxyester, oxyamido, axyl, an oxy derivative, heterocycle, vinyl and R³ may additionally represent C2-5 alkenyl, C2-5 alkynyl or azido each optionally substituted by one or more halogen, cyano, thiocyano, azido, cyclopropyl, acyl and/or phenyl; or phenylsulfonyloxy whereby any phenyl moiety may be substituted by one or more halogen, alkyl, haloalkyl, alkoxy, nitro, amino, and/or phenyl; most preferably methyl, ethyl, propyl, isopropyl, butyl, or isobutyl.

R^{2a}, R^{3a} and R^{4a} are hydrogen

R⁵, R⁶, R⁷ are the same or different and each is independently hydrogen, hydroxy, alkyl, aryl, heterocycle or oxy derivative; and

R⁸ is hydrogen, hydroxy, thiol, halogen, alkyl, aryl, heterocycle, alkylthio or thio derivative. Within these Compounds 1A, R¹ is preferably methyl, ethyl, propyl, isopropyl, butyl, or isobutyl; most preferably methyl, ethyl or n-propyl.

R² and R⁴ are preferably independently hydrogen or halogen or methyl, ethyl, propyl, isopropyl, butyl, isobutyl; and, most preferably, are each hydrogen.

R³ is preferably C1-5 alkyl, C2-5 alkenyl, C2-C5 alkynyl, cyclopropyl, azido, each optionally substituted by one or more halogen, cyano, thiocyano, azido, alkylthio, cyclopropyl, acyl and/or phenyl; phenyl; phenylsulfonyl; phenylsulfonyloxy, tetrazole, thiazole, thienyl, furyl, pyrrole, pyridine, whereby any phenyl moiety may be substituted by one or more halogen, alkyl, haloalkyl, alkoxy, nitro, amino, and/or phenyl; most preferably methyl, ethyl, propyl, isopropyl, butyl, or isobutyl.

X is preferably —COOH or —COOMe or —COOEt or —CONH₂; most preferably —CONH₂.

A further particular group of compounds of formula II (Compounds 1B) comprises those wherein,

X is -CA¹NH₂, -CA¹NHCH₃ or -CA¹N(CH₃)₂;

R¹ is alkyl or phenyl;

R³ is alkyl, alkenyl, allynyl, cyano, isothiocyanato, ether, carboxyl, amido, aryl, heterocycle; or

R³ is CH₂R¹⁰ wherein R¹⁰ is hydrogen, cycloalkyl, oxyester, oxyalkylsulfonyl, oxyarylsufonyl, aminoalkylsulfonyl, aminoarylsulfonyl, nitrooxy, cyano, isothiocyanato, azido, alkylthio, arylthio, alkylsulfinyl, alkylsulfonyl, heterocycle, aryloxy, alkoxy or trifluoroethyl;

R^{3a} is hydrogen, alkyl or aryl (especially with the proviso that when R^{3a} is hydrogen, R³ other than methyl);

or R³R^{3a} form a cycloalkyl;

and R², R^{2a}, R⁴ and R^{4a} are each hydrogen.

Within the compounds of formula II,

R¹ is preferably alkyl especially C1-12- more particularly C1-6-alkyl and is most preferably ethyl;

R², R^{2a}, R^{3a} and R^{4a} are preferably hydrogen;

R³ is preferably selected from hydrogen; C1-12-alkyl, especially C1-6-alkyl, each optionally substituted by one or more substituents selected from hydroxy, halogen, cyano, thiocyanato or alkoxy and attached to the ring either directly or via a thio, sulfinyl, sulfonyl, carbonyl or oxycarbonyl group and optionally additionally a C1-4-alkylene bridge, particularly methylene; C2-6-alkenyl or -alkynyl, especially C2-3-alkenyl or -alkynyl, each optionally substituted by one or more halogens; azido; cyano; amido; carboxy; triazolyl, tetrazolyl, pyrrolidinyl, pyridyl, 1-oxidopyridyl, thiomorpholinyl, benzodioxolyl, furyl, oxazolyl, pyrimidinyl, pyrrolyl, thiadiazolyl, thiazolyl, thienyl or piperazinyl each optionally substituted by one or more substituents selected from halogen, C1-6-alkyl and phenyl and attached to the ring either directly or via a carbonyl group or a C1-4-alkylene bridge, particularly methylene; naphthyl; or phenyl, phenylalkyl or phenylalkenyl each optionally substituted by one or more substituents selected from halogen, C1-6-alkyl, C1-6 haloalkyl, C1-6-alkoxy, C1-6-alkylthio, amino, azido, phenyl and nitro and each attached to the ring either directly or via an oxy, sulfonyl, sulfonyloxy, carbonyl or carbonyloxy group and optionally additionally a C1-4-alkylene bridge, particularly methylene;

R³a is preferably hydrogen or C1-4-alkyl;

R⁴ and R^{4a} are preferably, independently hydrogen, C1-4-alkyl, phenyl or benzyl.

A further group of compounds of formula II (Compounds 1C) comprises those in racemic form wherein, when X is —CONR⁵R⁶ and R¹ is hydrogen, methyl, ethyl or propyl, then substitution on the pyrrolidine ring is other than mono-, di-, or tri-methyl or mono-ethyl.

A further group of compound of formula II (Compounds 1D) comprises those in racemic form wherein, when X is —CONR⁵R⁶ and R¹ is hydrogen or C1-6-alkyl, C2-6-alkenyl or -alkynyl or cycloalkyl, each unsubstituted, then substitution in the ring is other than by alkyl, alkenyl or alkynyl, each unsubstituted.

A further particular group of compounds of formula II (Compounds 1E) comprises those wherein,

X is -CA¹NH₂;

R¹ is H;

R³ is azidomethyl, iodomethyl, ethyl optionally substituted by 1 to 5 halogen atoms, n-propyl optionally substituted by 1 to 5 halogen atoms, vinyl optionally substituted by one or two methyl, and/or 1 to 3 halogen atoms, acetylene optionally substituted by C1-4-alkyl, phenyl or halogen;

R^{3a} is hydrogen or halogen, preferably fluorine;

and R², R^{2a}, R⁴ and R^{4a} are each hydrogen;

as their racemates or in enantiomerically enriched form, preferably the pure enantiomers.

A further particular group of compounds of formula II (Compounds 1F) comprises those wherein,

X is -CA¹NH₂;

R¹ is H;

R³ is C1-6-alkyl, C2-6-alkenyl or C2-6-alkynyl optionally substituted by azido, oxynitro, 1 to 6 halogen atoms;

R^{3a} is hydrogen or halogen, preferably fluorine;

and R², R^{2a}, R⁴ and R^{4a} are each hydrogen;
as their racemates or in enantiomerically enriched form, preferably the pure enantiomers.

In all the above mentioned scopes when the carbon atom to which R¹ is attached is asymmetric it is preferably in the “S”-configuration.

The “pharmaceutically acceptable salts” according to the invention include therapeutically active, non-toxic base and acid salt forms which the compounds of formula II are able to form.

The acid addition salt form of a compound of formula II that occurs in its free form as a base can be obtained by treating the free base with an appropriate acid such as an inorganic acid, for example, a hydrohalic such as hydrochloric or hydrobromic, sulfuric, nitric, phosphoric and the like; or an organic acid, such as, for example, acetic, hydroxyacetic, propanoic, lactic, pyruvic, malonic, succinic, maleic, fumaric, malic, tartaric, citric, methanesulfonic, ethanesulfonic, benzenesulfonic, p-toluenesulfonlc, cyclamic, salicylic, p-aminosalicylic, pamoic and the like.

The compounds of formula II containing acidic protons may be converted into their therapeutically active, non-toxic base addition salt forms, e.g. metal or amine salts, by treatment with appropriate organic and inorganic bases. Appropriate base salt forms include, for example, ammonium salts, alkali and earth alkaline metal salts, e.g. lithium, sodium, potassium, magnesium, calcium salts and the like, salts with organic bases, e.g. N-methyl-D-glucamine, hydrabamine salts, and salts with amino acids such as, for example, arginine, lysine and the like.

Conversely said salt forms can be converted into the free forms by treatment with an appropriate base or acid.

Compounds of the formula II and their salts can be in the form of a solvate, which is included within the scope of the present invention. Such solvates include for example hydrates, alcoholates and the like.

Many of the compounds of formula II and some of their intermediates have at least one stereogenic center in their structure. This stereogenic center may be present in a R or a S configuration, said R and S notation is used in correspondence with the rules described in Pure Appl. Chem., 45 (1976) 11-30.

The invention also relates to all stereoisomeric forms such as enantiomeric and diastereoisomeric forms of the compounds of formula II or mixtures thereof (including all possible mixtures of stereoisomers).

Furthermore certain compounds of formula II which contain alkenyl groups may exist as Z (zusammen) or E (entgegen) isomers. In each instance, the invention includes both mixture and separate individual isomers.

Multiple substituents on the pyrrolidone ring can also stand in either cis or trans relationship to each other with respect to the plane of the pyrrolidone ring.

Some of the compounds of formula I may also exist in tautomeric forms. Such forms although not explicitly indicated in the above formula are intended to be included within the scope of the present invention.

With respect to the present invention reference to a compound or compounds is intended to encompass that compound in each of its possible isomeric forms and mixtures thereof unless the particular isomeric form is referred to specifically.

The preferred active compounds of formula II are the following: (2S)-2-[(4S)-4-(2,2-difluorovinyl)-2-oxopyrrolidinyl]butanamide; (2S)-2-[(4R)-2-oxo-4-propylpyrrolidinyl]butanamide: and (2S)-2-[(4S)-2-oxo-4-propylpyrrolidinyl]butanamide.

The present invention concerns also a pharmaceutical composition for the treatment or the prevention of dyskinesia comprising a therapeutically effective amount of an active compound as described above and a pharmaceutically acceptable carrier. In particular, it also concerns a pharmaceutical composition for the treatment or prevention of movement disorders or dyskinesia comprising a therapeutically effective amount of an active compound which is (S)-(−)-α-ethyl-2-oxo-1-pyrrolidineacetamide and a pharmaceutically acceptable carrier.

The present invention requires administration of an effective dose of the active compound for the treatment and/or the prophylaxis of movement disorders or dyskinesia. The dose required in accordance with the invention should be sufficiently high to permit the relief of movement disorders or dyskinesia. Pharmaceutical compositions comprising the active compound can, for example, be administered orally or parenterally, i.e., intravenously, intramuscularly or subcutaneously, intrathecally.

Pharmaceutical compositions which can be used for oral administration can be solids or liquids and can, for example, be in the form of tablets, pills, dragees, gelatin capsules, solutions, syrups, and the like.

To this end, the active compound can be used mixed with an inert diluent or a non-toxic pharmaceutically acceptable vehicle such as starch or lactose, for example. Optionally, these pharmaceutical compositions can also contain a binder such as microcrystalline cellulose, gum tragacanth or gelatine, a disintegrant such as alginic acid, a lubricant such as magnesium stearate, a glidant such as colloidal silicon dioxide, a sweetener such as sucrose or saccharin, or colouring agents or a flavouring agent such as peppermint or methyl salicylate. They also comprise compositions which can release the active substance in a controlled manner. Pharmaceutical compositions which can be used for parenteral administration are in the pharmaceutical forms which are known for this mode of administration and are in the form of aqueous or oily solutions or suspensions generally contained in ampoules, disposable syringes, glass or plastics vials or infusion containers.

In addition to the active compound, these solutions or suspensions can optionally also contain a sterile diluent such as water for injection, a physiologic saline solution, oils, polyethylene glycols, glycerine, propylene glycol or other synthetic solvents, antibacterial agents such as benzyl alcohol, antioxidants such as ascorbic acid or sodium bisulphite, chelating agents such as ethylene diamine-tetra-acetic acid, buffers such as acetates, citrates or phosphates and agents for adjusting the osmolarity, such as sodium chloride or dextrose.

These pharmaceutical forms are prepared using methods which are routinely used by pharmacists.

The percentage of active material in the pharmaceutical compositions can fall within a wide range of concentrations and depends on a variety of factors such as the patient's sex, age, weight and medical condition, as well as on the method of administration. Thus the quantity of active product in compositions for oral administration is at least 0.5% by weight and can be up to 80% by weight with respect to the composition weight.

In compositions for parenteral administration, the quantity of active material present is at least 0.5% by weight and can be up to 33% by weight with respect to the composition weight. For the preferred parenteral compositions, the dosage unit is in the range 0.5 mg to 5.000 mg of active product.

The daily dose can fall within a wide range of dosage units of active product, and is generally in the range of 0.01 to 100 mg/kilogram (kg). However, it should be understood that the specific doses can be adapted to particular cases depending on the individual requirements, at the physician's discretion.

The present invention concerns also a use of the pharmaceutical composition, described above, for the treatment of a patient suffering from a disease chosen among Huntington's disease, Parkinson's disease, and Schizophrenia, or for the treatment of patients exposed to chronic dopamine replacement therapy, or to chronic treatment with neuroleptics.

An active compound having formula II or the compound (S)-(−)-α-ethyl-2-oxo-1-pyrrolidineacetamide obviate or mitigate dyskinesia when used as a monotherapy or given in combination with other treatments which also reduce dyskinesia (e.g. μ-opioid receptor antagonists, alpha2-adrenoceptor-antagonists, cannabinold CB1-antagonists, NMDA receptor antagonists, adenosine A2a antagonists , H3-histamine receptor agonists, metabotropic Glutamate receptors antagonists, GP1 lesion/deep brain stimulation).

Therefore, the present invention relates also to a pharmaceutical composition comprising an active compound which is a 2-oxo-1-pyrrolidine derivatives having the formula II or a pharmaceutically acceptable salt thereof, or the compound (S)-(−)-α-ethyl-2-oxo-1-pyrrolidineacetamide, and at least one compound having anti-dyskinesia activity.

In another embodiment, the present invention relates to the use of (S)-(−)-α-ethyl-2-oxo-1-pyrrolidineacetamide for a manufacture of a medicament for treatment or prophylaxis of Parkinson's disease.

The present invention concerns also a pharmaceutical composition for the treatment of Parkinson's disease comprising a therapeutically effective amount of (S)-(−)-α-ethyl-2-oxo-1-pyrrolidineacetamide and a pharmaceutically acceptable carrier.

The present invention concerns also a pharmaceutical composition comprising (S)-(−)-α-ethyl-2-oxo-1-pyrrolidineacetamide and at least one compound having anti-parkinsonian activity. Examples of compounds having anti-parkinsonian activity are dopamine replacing agents (e.g. L-DOPA or dopamine agonists), anticholinergic drugs, amantadine, monoamine oxidase inhibitors. A particular example of the said compound is ropinirole.

The present invention relates to a method of selectively potentiating the therapeutic effect of a compound having anti-parkinsonian activity without increasing undesired side effects associated therewith which comprises co-administration of an amount of the said compound with an amount of (S)-(−)-α-ethyl-2-oxo-1-pyrrolidineacetamide effective in producing the desired therapeutic effect.

The following examples illustrate the invention without in any way limiting its scope.

### EXAMPLE 1

This study was designed to investigate whether levetiracetam has anti-dyskinetic activity in the 1-methyl-4-phenyl-1,2,3,6-tetrahydropyridine (MPTP)—lesioned marmoset model of Parkinson's disease. The effect of levetiracetam on L-3,4-dihydroxyphenylalanine (L-DOPA)-induced dyskinesias and alleviation of parkinsonism symptoms was investigated. The study was performed on seven adult marmosets (Callithrix jacchus) bred in a closed colony. The marmosets were rendered parkinsonian by subcutaneous injection of 2 mg/kg MPTP for 5 consecutive days. The marmosets were allowed to recover for 18 weeks until their parkinsonism became stable. The degree of activity and disability before and after MPTP treatment was assessed using a combination of scales that measure locomotor activity, mobility, bradykinesia and posture. Animals were treated with L-DOPA (12.5 mg/kg b.i.d. for 6 weeks) to prime them to elicit dyskinesia. After this time all animals demonstrated stable levels of dyskinesia when challenged with L-DOPA.

All drugs were administered orally in a volume of 5 ml/kg via a syringe in the animal's home cage. The animals were immediately transferred to the experimental cage (60 cm×55 cm×75 cm, with the perch 25 cm from floor of cage) for behavioural assessment. Vehicle was apple juice in all cases.

A battery of behavioural tests were performed:

1) Activity—a quantitative assessment of the amount of movement of the animal was obtained every 5 minutes for the duration of the experiment using computer-based activity monitors.

2) Parkinsonian disability—non-parametric measures based on the following scales

a) Range of movement score: 0=no movement, 1=movement of head on the floor of the cage, 2=movement of limbs, but no locomotion, on the floor of the cage, 3=movement of head or trunk on wall of cage or perch, 4=movement of limbs, but no locomotion, on wall of cage or perch, 5=walking around floor of cage or eating from hopper on floor, 6=hopping on floor of cage, 7=climbing onto wall of cage or perch, 8=climbing up and down the walls of the cage or along perch, 9=running, jumping, climbing between cage walls/perch/roof, uses limbs through a wide range of motion and activity.

b) Bradykinesia score: 0=normal speed and initiation of movement, 1=mild slowing of movement, 2=moderate slowing, difficulty initiating and maintaining movement, marked freezing, 3=akinetic, unable to move, with prolonged freezing episodes

c) Postural abnormality score: 0=normal, upright, holds head up, normal balance 1=abnormal, crouched, face down, may lose balance.
d) Parkinsonian disability score: A combination of the mobility, bradykinesia and posture scores according to the formula [18—(Range of movement*2)+(Bradykinesia*3)+(posture*9)] to give a global parkinsonian disability rating.

3) Dyskinesia—non-parametric measures based on the following scale

Dyskinesia score: 0=Absent, 1=Mild, fleeting, 2=Moderate, not interfering with normal activity, 3=Marked, at times interfering with normal activity, 4=Severe, continuous, replacing normal activity.

Behaviour was assessed for 6 hours post drug administration. Behavioural test 1, activity, was assessed every 5 minutes for 6 hours post drug administration. Behavioural tests 2 and 3, parkinsonian disability and dyskinesia, respectively, were assessed for 10 minutes every 30 minutes over the course of 6 hours, by post hoc analysis of video-recordings by an observer blinded to the treatment. The score given in each 10 minutes time period represents the maximum score achieved during that time period.

Table 1 outlines the randomised treatment schedule i.e. three doses of levetiracetam drug in combination with a single dose of L-DOPA. The actions of each of these three combination therapies were compared with that of L-DOPA plus the appropriate vehicle. Thus, a total of four treatments were given.

TABLE 1
Randomised treatment schedule

| | animal number | | | | | | |
|---|---|---|---|---|---|---|---|
| Date | 1 | 2 | 3 | 4 | 5 | 6 | 7 |
| Day 1 | V | D1 | D2 | D1 | D2 | D3 | V |
| Day 4 | D1 | D3 | V | D3 | V | D2 | D3 |
| Day 6 | D3 | D2 | D1 | D2 | D1 | V | D1 |
| Day 8 | D2 | V | D3 | V | D3 | D1 | D2 |

V = L-DOPA + vehicle
D1 = L-DOPA (12 mg/kg) + levetiracetam (13 mg/kg)
D2 = L-DOPA (12 mg/kg) + levetiracetam (30 mg/kg)
D3 = L-DOPA (12 mg/kg) + levetiracetam (60 mg/kg)

L-DOPA (12 mg/kg) alone reversed parkinsonian symptoms. The alleviation of parkinsonian symptoms was accompanied by severe dyskinesia.

Dyskinesia was significantly reduced following the combined treatment for the first hour post drug administration (p<0.01, p<0.05 and p<0.01 for 13 mg/kg, 30 mg/kg and 60 mg/kg, respectively; Friedman test followed by Dunn's multiple comparison's test). In contrast, there were no significant differences in disability scores for the first hour post drug administration (p>0.05 for 13 mg/kg, 30 mg/kg and 60 mg/kg; Friedman test followed by Dunn's multiple comparison's test). Co-administration of levetiracetam (13 to 60 mg/kg) and L-DOPA (12 mg/kg) reversed parkinsonian symptoms to the same magnitude, at peak effect, as L-DOPA (12 mg/kg) monotherapy. There were no significant differences in dyskinesia or disability scores at any time-point after one hour post drug administration (p>0.05 for 13 mg/kg, 30 mg/kg and 60 mg/kg; Friedman test followed by Dunn's multiple comparison's test).

Combined levetiracetam (13-60 mg/kg) and L-DOPA (12 mg/kg) treatment had the same maximal anti-parkinsonian action compared to L-DOPA monotherapy.

Combined levetiracetam (13-60 mg/kg) and L-DOPA (12 mg/kg) treatment was associated with less significantly dyskinesia, during the first hour post drug administration, than L-DOPA monotherapy.

In combination with L-DOPA, levetiracetam had a significant advantage over L-DOPA monotherapy.

The major benefit of levetiracetam was a reduction in L-DOPA-induced dyskinesia during the first hour post drug administration. This reduction in dyskinesia was seen without a reduction in anti-parkinsonian efficacy.

Thus, the clinical benefit for levetiracetam may be as an adjunctive therapy to reduce dyskinesia in parkinson patients exposed to chronic dopamine replacement therapy, in schizophrenia patients exposed to chronic neuroleptic treatment and in patients with Huntington's disease.

### EXAMPLE 2

This study was designed to investigate whether the compound ((2S)-2-[(4S)-4-(2,2-difluorovinyl)-2-oxopyrrolidinyl]butanamide) (named compound A) has anti-dyskinetic activity in the 1-methyl-4-phenyl-1,2,3,6-tetrahydropyridine (MPTP)—lesioned marmoset model of Parkinson's disease. The effect of the compound A on L-3,4-dihydroxyphenylalanine (L-DOPA)-induced dyskinesias and alleviation of anti parkinsonism symptoms was investigated.

The study was performed on nine adult marmosets (Callithrix jacchus) bred in a closed colony. The marmosets were rendered parkinsonian by subcutaneous injection of 2 mg/kg MPTP for 5 consecutive days. The marmosets were allowed to recover for 18 weeks until their parkinsonism became stable. The degree of activity and disability before and after MPTP treatment was assessed using a combination of scales that measure locomotor activity, mobility, bradykinesia and posture. Animals were treated with L-DOPA (13.9+/−0.8 mg/kg b.i.d. for 6 weeks) to prime them to elicit dyskinesia. After this time all animals demonstrated stable levels of dyskinesia when challenged with L-DOPA.

All drugs were administered orally in a volume of 5 ml/kg via a syringe in the animal's home cage. The animals were immediately transferred to the experimental cage (60 cm×55 cm×75 cm, with the perch 25 cm from floor of cage) for behavioural assessment. Vehicle was apple juice in all cases.

A battery of behavioural tests were performed:

1) Activity—a quantitative assessment of the amount of movement of the animal was obtained every 5 minutes for the duration of the experiment using computer-based activity monitors.

2) Parkinsonian disability—non-parametric measures based on the following scales

a) Range of movement score: 0=no movement, 1=movement of head on the floor of the cage, 2=movement of limbs, but no locomotion, on the floor of the cage, 3=movement of head or trunk on wall of cage or perch, 4=movement of limbs, but no locomotion, on wall of cage or perch, 5=walking around floor of cage or eating from hopper on floor, 6=hopping on floor of cage, 7=climbing onto wall of cage or perch, 8=climbing up and down the walls of the cage or along perch, 9=running, jumping, climbing between cage walls/perch/roof, uses limbs through a wide range of motion and activity.

b) Bradykinesia score: 0=normal speed and initiation of movement, 1=mild slowing of movement, 2=moderate slowing, difficulty initiating and maintaining movement, marked freezing, 3=akinetic, unable to move, with prolonged freezing episodes

c) Postural abnormality score: 0=normal, upright, holds head up, normal balance, 1=abnormal, crouched, face down, may lose balance.

d) Parkinsonian disability score: A combination of the mobility, bradykinesia and posture scores according to the formula [18—(Range of movement*2)+(Bradykinesia*3)+(posture*9)] to give a global parkinsonian disability rating.

3) Dyskinesia—non-parametric measures based on the following scale

Dyskinesia score: 0=Absent, 1=Mild, fleeting, 2=Moderate, not interfering with normal activity, 3=Marked, at times interfering with normal activity, 4=Severe, continuous, replacing normal activity.

Behaviour was assessed for 6 hours post drug administration. Behavioural test 1, activity, was assessed every 5 minutes for 6 hours post drug administration. Behavioural tests 2 and 3, parkinsonian disability and dyskinesia, respectively, were assessed for 10 minutes every 30 minutes over the course of 6 hours, by post hoc analysis of video-recordings by an observer blinded to the treatment. The score given in each 10 minutes time period represents the maximum score achieved during that time period.

Four doses of compound A drug (1 mg/kg, 3 mg/kg, 10 mg/kg and 30 mg/kg) in combination with a single dose of L-DOPA were tested using a randomized treatment schedule. The actions of each of these four combination therapies were compared with that of L-DOPA plus the appropriate vehicle. Thus, a total of five treatments were given. L-DOPA alone reversed parkinsonian symptoms. The alleviation of parkinsonian symptoms was accompanied by dyskinesia.

At the doses of 10 mg/kg and 30 mg/kg of compound A, dyskinesia was significantly reduced following the combined treatment with L-DOPA for the first hour post drug administration (p>0.05 for 1 mg/kg and 3 mg/kg, p<0.05 for 10 mg/kg and 30 mg/kg; Friedman test followed by Dunn's multiple comparison's test). In contrast, there were no significant differences in disability scores for the first hour post drug administration (p>0.05 for 1 mg/kg, 3 mg/kg, 10 mg/kg and 30 mg/kg; Friedman test followed by Dunn's multiple comparison's test). Co-administration of compound A (1 to 30 mg/kg) and L-DOPA (13.9+/−0.8 mg/kg) reversed parkinsonian symptoms to the same magnitude, at peak effect, as L-DOPA (13.9+/−0.8 mg/kg) monotherapy. There were no significant differences in dyskinesia or disability scores at any time-point after one hour post drug administration (p>0.05 for 1 mg/kg, 3 mg/kg, 10 mg/kg and 30 mg/kg; Friedman test followed by Dunn's multiple comparison's test).

Combined compound A (1-30 mg/kg) and L-DOPA (13.9+/−0.8 mg/kg) treatment had the same maximal anti-parkinsonian action compared to L-DOPA monotherapy.

Combined compound A (10 and 30 mg/kg) and L-DOPA (13.9+/−0.8 mg/kg) treatment was associated with less significantly dyskinesia, during the first hour post drug administration, than L-DOPA monotherapy.

In combination with L-DOPA, compound A had a significant advantage over L-DOPA monotherapy.

The major benefit of compound A was a reduction in L-DOPA-induced dyskinesia during the first hour post drug administration. This reduction in dyskinesia was seen without a reduction in anti-parkinsonian efficacy.

Thus, the clinical benefit for compound A may be as an adjunctive therapy to reduce dyskinesia in parkinson patients exposed to chronic dopamine replacement therapy, in schizophrenia patients exposed to chronic neuroleptic treatment and in patients with Huntington's disease.

### EXAMPLE 3

This study was designed to investigate whether Levetiracetam has a potential as an adjunctive anti-parkinsonian agent to dopamine replacement therapy in the 1-methyl-4-phenyl-1,2,3,6-tetrahydropyridine (MPTP)-lesioned marmoset model of Parkinson's disease. The effect of Levetiracetam on Ropinirole alleviation of parkinsonism symptoms was investigated.

The study was performed on six adult marmosets (Callithrix jacchus; 4 female, 2 male). The marmosets were rendered parkinsonian by subcutaneous injection of 2 mg/kg MPTP for 5 consecutive days. The marmosets were allowed to recover for 18 weeks until their parkinsonism was stable. The degree of activity and disability before and after MPTP treatment were assessed using a combination of scales that measure locomotor activity, mobility, bradykinesia and posture. Animals were treated with L-DOPA 12 mg/kg b.i.d. for 6 weeks. After this time, animals were used for assessment of potential symptomatic antiparkinsonian therapy. All drugs were administered orally in a volume of 5 ml/kg via a syringe in the animal's home cage. The animals were immediately transferred to an experimental cage (60 cm×55 cm×75 cm, with the perch 25 cm from floor of cage) for behavioural assessment. Vehicle was apple juice in all cases. The doses were 3.75 mg/kg of Ropinirole in combination with Levetiracetam at 13, 30 and 60 mg/kg. Behaviour was assessed for 6 hours post drug administration.

A battery of behavioural tests was performed:

1) Activity—a quantitative assessment using computer-based activity monitors was obtained every 5 minutes for the duration of the experiment.

2) Parkinsonian disability—non-parametric measures based on the following scales:
a) Range of movement score: 0=no movement, 1=movement of head on the floor of the cage, 2=movement of limbs, but no locomotion, on the floor of the cage, 3=movement of head or trunk on wall of cage or perch, 4=movement of limbs, but no locomotion, on wall of cage or perch, 5=walking around floor of cage or eating from hopper on floor, 6=hopping on floor of cage, 7=climbing onto wall of cage or perch, 8=climbing up and down the walls of the cage or along perch, 9=running, jumping, climbing between cage walls/perch/roof, uses limbs through a wide range of motion and activity. The score given was the maximum achieved in each 10 minute observation period.
b) Bradykinesia score: 0=normal speed and initiation of movement, 1=mild slowing of movement, 2=moderate slowing, difficulty initiating and maintaining movement, marked freezing, 3=akinetic, unable to move, with prolonged freezing episodes. The score given was representative of behaviour over the observation period.
c) Postural abnormality score: 0=normal, upright, holds head up, normal balance, 1=abnormal, crouched, face down, may lose balance. The score given was representative of behaviour over the observation period.
d) Parkinsonian disability score: A combination of the mobility, bradykinesia and posture scores according to the formula [18—(Range of movement*2)+(Bradykinesia*3)+(Posture*9)] to give a global parkinsonian disability rating.

Behavioural test 1 (activity) was assessed every 5 minutes for 6 hours post drug administration. Behavioural tests 2 (parkinsonian disability) was assessed for 10 minutes every 30 minutes over the course of 6 hours, by post hoc analysis of video-recordings by an observer blinded to the treatment. The score given/achieved in each 10 minute time period was presented.

Range of movement score: 0=none, 3=low, 6=moderate, 9=high

Bradykinesia score: 0=none, 1=mild, 2=moderate, 3=severe

Postural abnormality score: 0=none, 0.5=mild, 1=severe

Parkinsonian disability score: 0=none, 9=mild, 18=moderate, 27=marked, 36=severe Cumulated data for parkinsonian disability, range of movement, bradykinesia and postural abnormalities were analysed with a non-parametric repeated measures one-way ANOVA (Friedman's test) followed by Dunn's multiple comparison test (Graphpad Prism version 3). Levetiracetam, administered at 13 and 30 mg/kg but not 60 mg/kg, significantly potentiated the alleviation of parkinsonism by Ropinirole (3.75 mg/kg). Thus, Levetiracetam, administered at 13 and 30 mg/kg significantly increased activity and “on-time” (all P<0.01; one-way repeated measures ANOVA followed by Dunnett's multiple comparison's test). Also, Levetiracetam administered at 13 mg/kg significantly reduced parkinsonian disability over the experiment as a whole and specifically during 3-4 hour time period (P<0.05; Friedman's test followed by Dunn's multiple comparison's test). Furthermore, Levetiracetam, administered at 30 mg/kg, significantly increased range of movement during the 0-1 hour time period (P<0.05; Friedman's test followed by Dunn's multiple comparison's test). In conclusion , the increase in general activity levels was accompanied by a significant reduction in parkinsonian disability and reflects an enhancement of the anti-parkinsonian actions afforded by Ropinirole. Furthermore, there was an enhancement of “on-time” by approximately 82% and 69% for 13 mg/kg and 30 mg/kg Levetiracetam, respectively. However, activity counts were still elevated at the end of the six hour experiment suggesting that observed “on-time” might have been greater if the experiment had not been terminated at six hours.

Levetiracetam may have potential as an anti-parkinsonian agent in combination with dopamine replacement therapy. The extension of “on-time” might represent a useful de novo therapy to delay the onset of dyskinesia.

1-15. (canceled)

16. A method for treating Parkinson's disease in a chronic dopamine replacement therapy using L-DOPA, which comprises administering to a patient in need thereof an effective amount of a 2-oxo-1-pyrrolidine derivative selected from the group consisting of (S)-(−)-α-ethyl-2-oxo-1-pyrolidine-acetamide and ((2S)-2-[(4S)-4-(2,2-difluorovinyl)-2-oxopyrrolidinyl]butanamide).

17. A method for treating Parkinson's disease in a chronic dopamine replacement therapy using L-DOPA, which comprises administering to a patient in need thereof an effective amount of a 2-oxo-1-pyrrolidine derivative selected from the group consisting of (S)-(−)-α-ethyl-2-oxo-1-pyrrolidine-acetamide and ((2S)-2-[(4S)-4-(2,2-difluorovinyl)-2-oxopyrrolidinyl]butanamide) in combination with either amantadine or ropinirole.

18. A pharmaceutical composition comprising (S)-(−)-α-ethyl-2-oxo-1-pyrrolidine-acetamide) and amantadine as well as a pharmaceutically acceptable carrier.

19. A pharmaceutical composition comprising (S)-(−)-α-ethyl-2-oxo-1-pyrrolidine-acetamide and ropinirole as well as a pharmaceutically acceptable carrier.

20. A pharmaceutical composition comprising ((2S)-2-[(4S)-4-(2,2-difluorovinyl)-2-oxopyrrolidinyl]butanamide and amantadine as well as a pharmaceutically acceptable carrier.

21. A pharmaceutical composition comprising ((2S)-2-[(4S)-4-(2,2-difluorovinyl)-2-oxopyrrolidinyl]butanamide and ropinirole as well as a pharmaceutically acceptable carrier.
